(19) United States
(12) Patent Application Publication
(10) Pub. No.: US 2005/0079156 A1
(43) Pub. Date: Apr. 14, 2005

(54) Method of treating endometreosis

(76) Inventors: Grace Wong, Brookline, MA (US); Aliza Eshkol, La Rippe (CH)

Correspondence Address: BROWDY AND NEIMARK, P.L.L.C.;624 NINTH STREET, NW, SUITE 300, WASHINGTON, DC, 20001-5303, US

(21) Appl. No.: 10/494,699

(22) PCT Filed: Oct. 29, 2002
(86) PCT No.: PCT/US02/34440

Related U.S. Application Data
(60) Provisional application No. 60/332,940, filed on Nov. 6, 2001.

(51) Int. Cl. A61K 38/21; A61K 31/56; A61K 31/138

(52) U.S. Cl. 424/85.6; 514/171; 514/651

(57) ABSTRACT

The present invention relates to the treatment of a woman for endometriosis comprising the administration of IFN-gamma, a cytokine antagonist and/or an anti-estrogenic agent.

### BACKGROUND OF THE INVENTION

Endometriosis is a disease affecting women in their reproductive years. The name comes from the word “endometrium,” which is the tissue that lines the inside of the uterus and builds up and sheds each month in the menstrual cycle. In endometriosis, endometrium-like tissue is found outside the uterus, in other areas of the body. In these locations outside the uterus, the endometrial tissue develops into what are called “nodules,” “tumors,” “lesions,” “implants,” or “growths.” These growths can cause pain, IFNertility, and other problems.

The most common locations of endometrial growths are in the abdomen involving the ovaries, fallopian tubes, the ligaments supporting the uterus, the area between the vagina and rectum, the outer surface of the uterus, and the lining of the pelvic cavity. Sometimes the growths are also found in abdominal surgery scars, on the intestines or in the rectum, on the bladder, vagina, cervix, and vulva (external genitals).

Endometrial growths have also been found outside the abdomen, in the lung, arm, thigh, and other locations, but these are uncommon. Endometrial growths are generally not malignant or cancerous. However, in recent decades there has been an increased frequency of malignancy occurring or being recognized in conjunction with endometriosis. Like the lining of the uterus, endometrial growths usually respond to the hormones of the menstrual cycle. They build up tissue each month, break down, and cause bleeding.

However, unlike the lining of the uterus, endometrial tissue outside the uterus has no way of leaving the body. The result is internal bleeding, degeneration of the blood and tissue shed from the growths, inflammation of the surrounding areas, and formation of scar tissue. Other complications, depending on the location of the growths, can be rupture of growths (which can spread endometriosis to new areas), the formation of adhesions, intestinal bleeding or obstruction (if the growths are in or near the intestines), interference with bladder function (if the growths are on or in the bladder), and other problems. Symptoms seem to worsen with time, though cycles of remission and reoccurrence are the pattern in some cases.

Symptoms:

The most common symptoms of endometriosis are pain before and during periods (usually worse than “normal” menstrual cramps), during or after sexual activity, infertility, and heavy or irregular bleeding. Other symptoms may include fatigue; painful bowel movements with periods; lower back pain with periods; diarrhea and/or constipation and other intestinal upset with periods. Some women with endometriosis have no symptoms. Infertility affects about 30-40% of women with endometriosis and is a common result with progression of the disease.

The amount of pain is not necessarily related to the extent or size of growths. Tiny growths (called“petechial”) have been found to be more active in producing prostaglandins, which may explain the significant symptoms that often seem to occur with small implants. Prostaglandins are substances produced throughout the body, involved in numerous functions, and thought to cause many of the symptoms of endometriosis.

Theories about the Cause of Endometriosis:

The cause of endometriosis is not known. A number of theories have been advanced but no one of them seems to account for all cases. One theory is the retrograde menstruation or transtubal migration theory that during menstruation some of the menstrual tissue backs up through the fallopian tubes, implants in the abdomen, and grows. Another theory suggests that the endometrial tissue is distributed from the uterus to other parts of the body through the lymph system or the blood system. A genetic theory suggests that it may be carried in the genes of certain families or that certain families may have predisposing factors to endometriosis.

Another theory suggests that remnants of tissue from when the woman was an embryo may later develop into endometriosis or that some adult tissues retain the ability they had in the embryo stage to transform into reproductive tissue under certain circumstances. Surgical transplantation has also been cited as a cause in cases where endometriosis is found in abdominal surgery scars, although it has also been found in such scars when direct accidental implantation seems unlikely.

Some experts on endometriosis believe all women experience some menstrual tissue backup and that an immune system problem and/or hormonal problem allows this tissue to take root and grow in women who develop endometriosis. In fact, estrogens are also known to promote the proliferation of normal endometrium. Chronic exposure to estrogens unopposed by progesterone can lead to the development of endometrial hyperplasia which predisposes to endometrial carcinoma. The incidence of endometrial cancer increases after menopause, especially in women receiving estrogen therapy without simultaneous treatment with progestins.

Treatment:

Treatment for endometriosis has varied over the years but no certain cure has yet been found. Hysterectomy and removal of the ovaries has been considered a “definitive” cure, but research has found such a high rate of continuation/recurrence. Painkillers are usually prescribed for the pain of endometriosis.

Current pharmacologic therapy for endometriosis requires hormonal suppression of the production of estrogen, so that the poor hormone environment blocks the growth of ectopic tissue. Hormonal treatments include estrogen and progesterone, progesterone alone, a testosterone derivative (danazol), and a new drug, GnRH, gonadotropin releasing hormone. Side effects are a problem for some women with all hormonal treatments.

The development of therapeutics with greater efficacy for the treatment of endometriosis is an on-going need.

### SUMMARY OF THE INVENTION

The present invention is based upon the unexpected discovery that interferon-gamma (i.e., IFN-γ) can inhibit the constitutive transcription and cytokine induced transcription of the aromatase gene in adipocytes and therefore provides methods of inhibiting estrogen production in these cells. Thus, the present invention provides novel methods for treating endometriosis.

In one embodiment, the invention provides a method of treating endometriosis in a woman in need thereof comprising administering to the woman a therapeutically effective amount of IFN-γ which, in turn inhibits aromatase mRNA transcription and subsequent estradiol production from adipocytes.

IFN-γuseful in the method of the present invention includes native IFN-γ, recombinant IFN-γ and IFN-γ that has been modified, for example, to increase its stability.

In another embodiment, the invention provides a method of treating endometriosis in a woman in need thereof comprising administering to the woman a therapeutically effective amount of a cytokine antagonist that inhibits cytokine induced aromatase mRNA transcription and subsequent estradiol production from adipocytes.

Cytokine antagonists useful in the method of present invention include soluble cytokine receptor molecules, anti-cytokine antibodies and compounds which prevent and/or inhibit cytokine receptor signaling. Proteins, muteins, protein-derived peptides, mimetics and small molecule drugs that inhibit estradiol induction by cytokines in adipocytes can also be used in the present invention. It is possible to use the cytokine antagonists alone or a combination with other cytokine antagonists.

Preferably, the cytokine antagonist is an inhibitor of a cytokine selected from a group consisting of tumor necrosis factor-related apoptosis-inducing ligand (TRAIL), OX40 Ligand (OX40L), lymphotoxin alpha (LT-α), interleukin-1-alpha (IL-1-α), interleukin-1 beta (IL-1-β), interleukin-6 (IL-6), interleukin-8 (IL-8), interleukin-11 (IL-11), leukemia inhibitory factor (LIF), Fas Ligand (FasL), oncostatin M, interferon alpha (IFN-α), interferon beta (IFN-β), insulin growth factor-1 (IGF-1), stem cell factor (SCF) and human growth hormone (GH). It is important to note that antagonists of interferon-gamma are not considered for the purposes of the present invention to be included as cytokine antagonists.

In another embodiment, IFN-γ is administered to the woman before, after, or simultaneously with a cytokine antagonist.

In another embodiment, IFN-γ and/or a cytokine antagonist is administered to the woman before, after, or simultaneously with some other anti-estrogen treatment methods.

The invention also features an article of manufacture including packaging material and a pharmaceutical agent contained therein that is therapeutically effective for treating or preventing endometriosis in a woman. The packaging material may include a label that indicates that the pharmaceutical agent can be used for treating and/or preventing endometriosis. The pharmaceutical agent includes an effective amount of IFN-γ or an effective amount of a cytokine antagonist. In one embodiment, the pharmaceutical agent includes a combination of an effective amount of IFN-γ and a cytokine antagonist.

In an alternate embodiment, the invention relates to compositions and kits comprising a first endometriosis treating or preventing agent including IFN-γ or a cytokine antagonist and a second therapeutic agent. The second therapeutic agent is not IFN-γ or a cytokine antagonist. These compositions are effective to treat or prevent endometriosis in a woman. Various classes of therapeutic agents, including anti-estrogenic agents such as aromatase inhibitors, may be used in the composition. In one embodiment, the first endometriosis treating or preventing pharmaceutical agent includes a combination of an effective amount of IFN-γ and a cytokine antagonist.

Other aspects of the invention are disclosed infra.

### BRIEF DESCRIPTION OF THE FIGURES

FIG. 1 illustrates that IFN-γ inhibits IL-1α and IL-1β induction of estradiol production in human adipocytes.

FIG. 2 illustrates the dose-dependent inhibitory effects of recombinant human IFN-γ on constitutive estradiol production in human adipocytes.

FIG. 3 illustrates that interferon gamma (IFN-γ) inhibits the transcription of aromatase mRNA when transcription is induced by interleukin-1β (IL-2β) or tumor necrosis factor (TNF). The lower panel shows transcription of a household gene, glycero-aldehyde phosphate dehydrogenase (GAPDH). Control lane shows the base level of aromatase mRNA expression in adipocytes.

FIG. 4 illustrates the differential effects of interferon (IFN) α, β, and γ on estradiol production from human adipocytes stimulated by a mixture of cytokines. IFN-γ inhibits stimulated production while IFN-α and IFN-β do not inhibit stimulated production of estradiol.

FIG. 5 illustrates that IFN-γ inhibits the stimulation of estradiol production in human adipocytes by a mixture of 15 Cytokines (IL-1a, IL-1b, TNF-α, TNF-β, IL-6, IL-8, IL-11, GH, IGF-1, LIF, SCF, FAS, TRAIL, OX40 L and oncostatin M, 0.01 ug/ml/each).

FIG. 6 illustrates that IFN-γ inhibits LPS stimulation of estradiol production in human adipocytes.

FIG. 7 illustrates that IFN-γ inhibits stimulation of estradiol production in human adipocytes by a mixture of PMA (5 ng/ml), LPS (10 ug/ml) and PGE2 (10 ug/ml).

FIG. 8 illustrates that the cytokines, tumor necrosis factor-related apoptosis-inducing ligand (TRAIL), OX40 Ligand (OX40L), interleukin-1-beta (IL-1-β), interleukin-6 (IL-6), interleukin-11 (IL-11), and leukemia inhibitory factor (LIF) all induce aromatase mRNA expression and the induction of aromatase mRNA expression by all these Cytokines is inhibited by IFN-β.

FIG. 9 illustrates that Cytokines (IL-6, IL-8, IL-11, OSM, GH and IGF-1) stimulate the production of estradiol in human preadipocytes.

FIG. 10 illustrates that monoclonal antibodies to Oncostatin M inhibit the induction of estradiol production inhuman adipocytes by Oncostatin M.

FIG. 11 illustrates that monoclonal antibodies to IL-11 inhibit the induction of estradiol production in human adipocytes by IL-11.

FIG. 12 illustrates that Interferon Alpha/beta Receptor (IFNAR) inhibits the IFN-β induction of estradiol production in human adipocytes.

FIG. 13 illustrates that IL-1 Receptor Antagonist (IL-1RA) inhibits the IL-1α and IL-1β induction of estradiol production in human adipocytes.

It is to be understood that both the foregoing general description and the following detailed description are merely exemplary of the invention, and are intended to provide an overview or framework for understanding the nature and character of the invention as it is claimed.

### DETAILED DESCRIPTION OF THE INVENTION

The present invention is based upon an unexpected discovery that interferon-gamma (IFN-γ) inhibits transcription of the aromatase gene in adipocytes. Aromatase is an enzyme that is required to convert a sex hormone precursor androstenedione into estrone which is thereafter modified into estrogen. When the function of aromatase enzyme is inhibited, decreased levels of estrogen can be produced from its precursors by the cells. Therefore, the present invention provides a method of blocking estrogen production by administering IFN-γ, which is useful in the treatment of endometriosis.

Interferon-gamma has non-specific antiviral, antiproliferative and in particular immunomodulatory effects. Its production in T-helper-lymphocytes is stimulated by mitogens and antigens. The effect of the expressed IFN-γ has not yet been precisely clarified, but is subject to intensive research. In particular, IFN-γ leads to the activation of macrophages and to the synthesis of histocompatability antigens of the class 2. In vitro, the activity of IFN-γ is normally determined as a reduction in the virus-induced cytopathic effect, which arises from treatment with interferon-gamma. Due to its antigen-non-specific antiviral, antiproliferative and immunomodulatory activity it is suitable as a human therapeutic agent, for example of kidney tumours and chronic granulomatosis. Clinical studies of various tumours are being carried out at present. The finding that IFN-γ inhibits constitutive and induced production of estradiol for adipocytes is quite unexpected.

All forms of human IFN-γ that are shown to be biologically active can be used according to the present invention. These forms include mature, pro, met and/or des(1-3) (also referred to as des-Cys-Tyr-Cys IFN-γ) form, whether obtained from natural source, chemically synthesized or produced by techniques of recombinant DNA technology. A complete description of the preparation of recombinant human IFN-γ (rhu IFN-γ) including its cDNA and amino acid sequences are disclosed, for example, in U.S. Pat. No. 4,727,138; U.S. Pat. No. 4,762,791; U.S. Pat. No. 4,925,793; U.S. Pat. No. 4,929,554; U.S. Pat. No. 5,582,824; U.S. Pat. No. 5,096,705; U.S. Pat. No. 4,855,238; U.S. Pat. No. 5,574,137; and U.S. Pat. No. 5,595,888. CysTyrCys-lacking recombinant human IFN-γ, including variously truncated derivatives are, for example, disclosed in U.S. Pat. No. 5,582,824. IFN-γ useful in the present invention includes variously glycosylated forms and other variants (e.g. amino acid sequence variants) and derivatives of such native (wild-type) IFN-γ, whether known in the art or becoming available in the future. Examples of such variants are alleles, and the products of site-directed mutagenesis in which residues are deleted, inserted and/or substituted (see, e.g. U.S. Pat. No. 5,582,824 referred to above). IFN-γ useful in the present invention is available from a wide variety of commercial sources and it is approved for the treatment of numerous indications.

The IFN-γ used according to the present invention may be from natural sources, but is preferably a recombinant product. IFN-γ useful according to the present invention also includes polypeptides or fragments thereof which have IFN-γ activity, and chimeric or mutant forms of IFN-γ in which sequence modifications have been introduced, for example to enhance stability, without affecting the nature of their biological activity, such as disclosed in U.S. Pat. No. 5,593,667, and U.S. Pat. No. 5,594,107 among others. For example, the IFN-γ useful in the present invention can be a recombinant human IFN-γ species (recombinant human interferon gamma-1b, rh IFN-γ-1b, containing 140 amino acids), which is the active ingredient of the commercial formulation, Actimmune® (InterMune, Inc., Brisbane, Calif.).

The present invention also is based on the finding that many cytokines induce the transcription of the aromatase gene and subsequently increase estradiol production in adipocytes. Therefore, the present invention provides methods of blocking adipocyte estrogen production by administering one or more cytokine antagonists, which are useful in the treatment of endometriosis.

The term cytokine, or immunocytokine, was used initially to separate a group of immunomodulatory proteins, called also immunotransmitters from other growth factors that modulate the proliferation and bioactivities of non-immune cells. Currently, however, the term cytokine is used as a generic name for a diverse group of soluble proteins and peptides which act as humoral regulators at nano- to picomolar concentrations and which, either under normal or pathological conditions, modulate the functional activities, such as enzyme production, of individual cells and tissues. These proteins also mediate interactions between cells directly and regulate processes taking place in the extracellular environment. Due of the involvement of cytokines in modulating immune responses they have been considered as potential therapeutic agents. However, it has been difficult to distinguish the effects Of cytokines, that may have different effects on different tumor types, from the effects that are mediated secondarily by other immune effector cells.

Most cytokines are glycoproteins with signal sequences which are secreted by cells using classical secretory pathways. Many genes encoding cytokines can give rise to a number of variant forms of cytokines by means of alternative splicing, yielding molecules with slightly different but biologically significant bioactivities. In many cases the expression patterns of different forms of cytokines or of members of a cytokine family are overlapping only partially, suggesting a specific role for each factor. While expression of cytokines is normally transient and can be regulated at all levels of gene expression, also constitutive expression has been observed. The expression of many cytokines also seems to be regulated differentially, depending on cell type and developmental age.

The term “cytokine antagonist” as used herein means any agent that inhibits the cytokine's aromatase mRNA transcription activity as determined, for example, by the method of Example 3. Examples of such agents include, but are not limited to, a neutralizing antibody, including polyclonal, monoclonal and humanized antibodies against cytokine or an antigenic fragment of a cytokine; an antisense oligonucleotide complementary to sequence or a fragment of a sequence of mRNA encoding a cytokine; a soluble cytokine receptor or a modified soluble cytokine receptor. For example, IL-6 can be inhibited using an IL-6 antibody, an IL-6 receptor antibody, or a gp 130 antibody disclosed in the U.S. Pat. No. 6,086,874; LIF, oncostatin M, and IL-6 may also be inhibited using heteromeric proteins comprising a soluble alpha specificity determining cytokine receptor component and the extracellular domain of a beta receptor component as described in U.S. Pat. No. 5,844,099. It is important to note that antagonists of IFN-γ are not considered for the purposes of the present invention to be included as cytokine antagonists.

The cytokine antagonists preferably inhibit aromatase mRNA transcription by at least 50% in an assay as in Example 3. More preferably the antagonist inhibits aromatase mRNA transcription by 75%, most preferably 95%. Additional antagonists can be identified and tested using, for example, the assay of Example 3.

The cytokine antagonist is preferably, an inhibitor of a cytokine selected from a group consisting of tumor necrosis factor-related apoptosis-inducing ligand (TRAIL), OX40 Ligand (OX40L), lymphotoxin alpha (LT-α), interleukin-1 alpha (IL-1-α), interleukin-1 beta (IL-1-β), interleukin-6 (IL-6), interleukin-8 (IL-8), interleukin-11 (IL-11), leukemia inhibitory factor (LIF) Fas Ligand (FasL), oncostatin M, interferon alpha (IFN-α), interferon beta (IFN-β), insulin growth factor-1 (IGF-1), stem cell factor (SCF) and human growth hormone (GH).

A further embodiment of the present invention is the use of IFN-γ or a cytokine antagonist together with a pharmaceutically acceptable carrier in the preparation of pharmaceutical compositions for the treatment and/or prevention of endometriosis in a woman. The pharmaceutical composition may also include an anti-estrogenic agent.

In another embodiment, interferon-gamma is administered to the woman before, after, or simultaneously with a cytokine antagonist.

In another embodiment, interferon-gamma and/or a cytokine antagonist is administered before, during or after an anti-estrogenic agent.

In embodiments of the present invention which combine cytokine anatgonists with IFN-γ and/or anti-estrogenic agents, the cytokine antagonist is preferably an inhibitor of the cytokines listed above, as well as, an inhibitor of Tissue Necrosis Factor (TNF), such as Tissue Necrosis Factor Binding Proteins (TBP) as described in the European Patent Applications EP 308,378; EP 398,327 and EP 433,900 and U.S. patent and U.S. patent application Ser. Nos. U.S. Pat. No. 5,359,037; U.S. Pat. No. 5,512,544; U.S. Pat. No. 5,695,953; U.S. Pat. No. 5,811,261; U.S. Pat. No. 5,981,701; U.S. Pat. No. 6,232,446; U.S. Pat. No. 6,262,239; and US20010019833A1.

Cytokine antagonists can exert their activity in one of two ways. First, antagonists can bind to or sequester the cytokine molecule itself with sufficient affinity and specificity to substantially neutralize the cytokine epitope responsible for cytokine receptor binding (hereinafter termed sequestering antagonists). Alternatively, cytokine antagonists can inhibit cytokine signaling pathway activated by the cell surface receptor after cytokine binding (hereinafter termed “signaling antagonists”). Both groups of antagonists are useful, either alone or together, in the therapy of estrogen response breast cancer, according to the present invention.

Cytokine antagonists are easily identified and rated by routine screening of candidates for their effect on the activity of native cytokine on susceptible cell lines in vitro, for example human B cells, in which cytokines can cause proliferation and Ig secretion. The assay contains cytokine formulation at varying dilutions of candidate antagonist, e.g. from 0.1 to 100 times the molar amount of cytokine used in the assay, and controls with no cytokine or only antagonist (Tucci et al., Effects of eleven Cytokines and of IL-1 and tumor necrosis factor inhibitors in a human B cell assay. J Immunol. May 1, 1992; 148(9):2778-84).

Sequestering antagonists are the preferred cytokine antagonists according to the present invention. Among sequestering antagonists, those polypeptides that bind cytokine with high affinity and possess low immunogenicity are preferred. Soluble cytokine receptor molecules and neutralizing antibodies to cytokines are particularly preferred. Truncated forms of these receptors, comprising the extracellular domains of the receptors or functional portions thereof, are more particularly preferred antagonists according to the present invention. Derivatives, fragments, regions and biologically active portions of the receptor molecules functionally resemble the receptor molecules that can be used in the present invention. Such biologically active equivalent or derivative of the receptor molecule refers to the portion of the said polypeptide, or of the sequence encoding the receptor molecule, that is of sufficient size and able to bind cytokine with such an affinity that the interaction with the membrane-bound cytokine receptor is inhibited or blocked.

Cytokine receptor multimeric molecules and cytokine immunoreceptor fusion molecules, and derivatives or portions thereof, are additional examples of receptor molecules useful in the methods of the present invention. Cytokine receptor multimeric molecules useful in the present invention comprise all or a functional portion of the extracellular domain of two or more cytokine receptors linked via one or more polypeptide linkers. The multimeric molecules can further comprise a signal peptide of a secreted protein to direct expression of the multimeric molecule.

Cytokine immunoreceptor fusion molecules useful in the methods of the present invention comprise at least one portion of one or more immunoglobulin molecules and all or a functional portion of one or more cytokine receptors. These immunoreceptor fusion molecules can be assembled as monomers, or hetero- or homo-multimers. The immunoreceptor fusion molecules can also be monovalent or multivalent.

Another class of sequestering antagonists useful in the method of the present invention is represented by the anti-cytokine antibodies, including monoclonal, chimeric humanized, and recombinant antibodies and fragment thereof which are characterized by high affinity binding to cytokine in vivo and low toxicity. The antibodies which can be used in the invention are characterized by their ability to treat patients for a period sufficient to have good to excellent regression of endometriotic lesions, alleviation of symptoms and low toxicity. Neutralizing antibodies are readily raised in animals such as rabbits or mice by immunization with cytokine.

Immunized mice are particularly useful for providing sources of B cells for the manufacture of hybridomas, which in turn are cultured to produce large quantities of anti-cytokine monoclonal antibodies. Chimeric antibodies are immunoglobulin molecules characterized by two or more segments or portions derived from different animal species. Generally, the variable region of the chimeric antibody is derived from a non-human womanian antibody, such as murine monoclonal antibody, and the immunoglobulin constant region is derived from a human immunoglobulin molecule. Preferably, both regions and the combination have low immunogenicity as routinely determined (Elliott et al., “Randomised Double-blind Comparison of Chimeric Monoclonal Antibody to Tumour Necrosis Factor .alpha. (cA2) versus Placebo in Rheumatoid Arthritis”, Lancet, 344:1105-1110 (1994). Humanized antibodies are immunoglobulin molecules created by genetic engineering techniques in which the murine constant regions are replaced with human counterparts while retaining the murine antigen binding regions. The resulting mouse-human chimeric antibody should have reduced immunogenicity and improved pharmacokinetics in humans.

As use herein, “anti-estrogenic agent” includes the used of estrogen receptor modulators as described, for example in U.S. Pat. No. 6,300,367 including, but not limited to raloxifene, droloxifene, toremifene, 4′-iodotamoxifen, and idoxifene is co-administered with at least one isoflavone selected from genistein, daidzein, biochanin A, formononetin, and their naturally occuring glucosides and glucoside conjugates.

Examples of the anti-estrogenic agents include, but are not limited to 2-phenyl-3-benzothiophenes and 1-(alkylaminoethoxy phenyl)-1-phenyl-2-phenylbut-1-enes represented by raloxifene and tamoxifen; 4-hydroxytamoxifen; clomiphene; nafoxidine (Upjohn & Co., 700 Portage Road, Kalamazoo, Mich.); non-steroidal sulfatase inhibitor compounds as described in U.S. Pat. No. 5,567,831; derivatives of estra 1,3,5 (10)triene-17-one, 3-amino compounds as in U.S. Pat. No. 5,571,933; anti-estrogenic steroid sulfatase inhibitors as described in U.S. Pat. No. 6,288,050. Other anti-estrogenic agents that are contemplated in the present invention are toremifene, droloxifene, TAT-59, idoxifene, EM 139, clomiphene, MER-25, DES, nafoxidene, CP-336,156, GW5638, LY139481, LY353581, zuclomiphene, enclomiphene, ethamoxytriphetol, delmadinone acetate, bisphosphonate, and the like.

Several steroidal anti-estrogens have been synthesized which lack estrogenic activity. Included among these are ICI 164,384, ICI 182,780 and RU 58668. See, e.g.: Wakeling et al. J Steroid Biochem. 31:645-653 (1988), which pertains to ICI 164,384; Wakeling et al., Cancer Res. 51:3867-3873 (1991), and Wakeling et al., J. Steroid Biochem. Molec. Biol. 37:771-774 (1990), which pertain to ICI 182,780; and Van de Velde et al., Ann. N.Y. Acad. Sci. 761:164-175 (1995), Van de Velde et al., Pathol. Biol 42:30 (1994), and Nique et al., Drugs Future 20:362-366 (1995), which relate to RU 58668.

Anti-estrogenic agents as provided in the U.S. Pat. No. 6,281,205 are also contemplated in the present invention. Anti-estrogenic agents useful in the present invention also include estrogen receptor modulators as described, for example in U.S. Pat. No. 6,300,367 including, but not limited to raloxifene, droloxifene, toremifene, 4′-iodotamoxifen, and idoxifene is co-administered with at least one isoflavone selected from genistein, daidzein, biochanin A, formononetin, and their naturally occuring glucosides and glucoside conjugates.

Other compounds included as anti-estrogenic agents are aromatase inhibitors. These inhibitors refer to chemical compounds or polypeptides that block or inhibit the activity of aromatase which is an enzyme that converts androgens to estrogens. Examples of aromatase inhibitors include letrozole, anastrozole, vorozole and exemestane. (Journal of Endocrinology, February 2000: 164(2): 225-238; and Journal of Steroid Biochemistry and Molecular Biology, April 1997: 61(3-6): 157-166).

The invention also features an article of manufacture including packaging material and a pharmaceutical agent contained therein that is therapeutically effective for treating or preventing endometriosis in a woman. The packaging material may include a label that indicates that the pharmaceutical agent can be used for treating and/or preventing endometriosis. The pharmaceutical agent includes an effective amount of IFN-γ or an effective amount of a cytokine antagonist. In one embodiment, the pharmaceutical agent includes a combination of an effective amount of IFN-γ and a cytokine antagonist.

In an alternate embodiment, the invention relates to compositions and kits comprising a first endometriosis treating or preventing agent including IFN-γ or a cytokine antagonist and a second therapeutic agent. The second therapeutic agent is not IFN-γ or a cytokine antagonist. These compositions are effective to treat or prevent endometriosis in a woman. Various classes of therapeutic agents may be used in the composition. In one embodiment, the first endometriosis treating or preventing pharmaceutical agent includes a combination of an effective amount of IFN-γ and a cytokine antagonist.

Pharmaceutical Compositions

Interferon-gamma, cytokine anatgonists and anti-estrogenic agents (also referred to herein as “active compounds”) of the invention, and derivatives, fragments, analogs and homologs thereof, can be incorporated into pharmaceutical compositions suitable for administration. Such compositions typically comprise the nucleic acid molecule, protein, antibody or chemical and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration and do not interfer with the effectiveness of the active compounds. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., Interferon-gamma, cytokine antagonists and/or anti-estrogenic agents) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to IFNected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics or the active compound and the particular therapeutic effect to be achieved, and the limitations inherent in the art of compounding such an active compound for the treatment of individuals.

The pharmaceutical compositions can be included in a container, pack, or dispenser together with instructions for administration.

The present invention will now be illustrated by the example, which is not intended to be limiting in anyway, and makes reference to the figures within.

### EXAMPLE 1

### IFN-γ Inhibited IL-1 Induction of Estradiol Production in Human Adipocytes

Human subcutaneous cultured adipocytes, catalog nos. SP-1012, SP-1024, SP-1096, SP-75, or SP-25 were purchased from Zen-Bio, Inc. (Research Triangle Park, N.C.) and cultured according to the manufacturer's instructions.

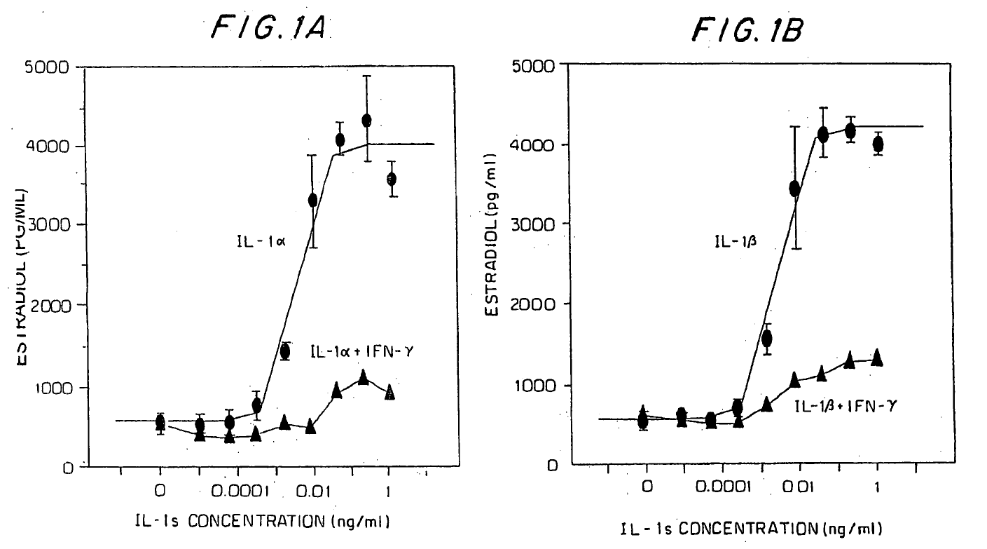

Human adipocytes (approximately 100,000 cells/well) were cultured for 24 hr in the presence or absence of different doses of IL-1α and IL-1β (as indicated) with and without IFN-γ (0.1 ug/ml, IL-1α, IL-1β and IFN-γ were purchased from R&D). The amount of estradiol release in the conditioned media was determined by radioimmunoassay for estradiol using the Active™ Estradiol EIA kit according to the manufacturer's instructions (catalog no. DSL-10-4300, Diagnostic Systems Laboratories, Inc., Webster, Tex.). Each value represents the mean±S.E.M. of triplicate wells. Similar results were reproducible in 10 additional experiments using cells derived from either male or female patients. As indicated by filled ovals in FIG. 1, both IL-1α and IL-1β, significantly increased estradiol production. However, as also illustrated in FIG. 1, when IFN-γ was given to the cells, the filled triangles show that IFN-γ inhibits IL-1α and IL-1β induced estradiol production in human adipocytes.

### EXAMPLE 2

### Dose-Dependent Inhibitory Effects of Recombinant Human IFN-γ on Constitutive Estradiol Production in Human Adipocytes

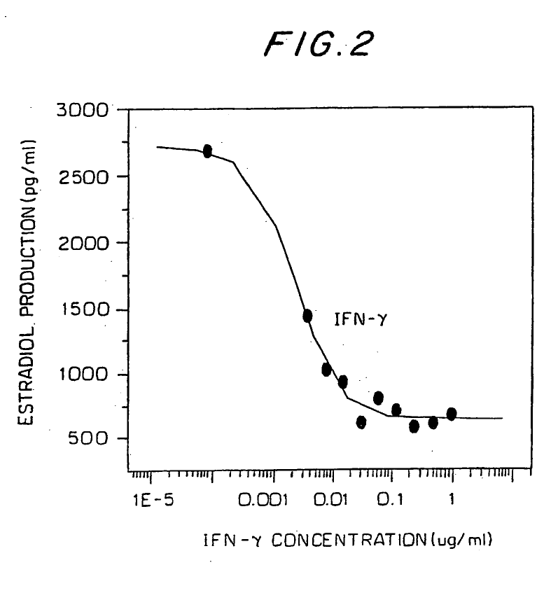

Human adipocytes (100,000 cells/well) were cultured for 72 hr with and without increasing doses of IFN-γ (0.0001 to 1 ug/ml, purchased from R&D). The amount of estradiol in the conditioned media was determined by radioimmunoassay as described in Example 1. Each value represents the mean±S.E.M. of triplicate wells. Similar results were reproducible in 3 additional experiments using cells derived from female patients. The results show, as illustrated in FIG. 2, IFN-γ has a dose-dependent inhibitory effect on constitutive estradiol production from human adipocytes.

### EXAMPLE 3

### IFN-γ Inhibited Constitutive and IL-1b Induction of Aromatase mRNA in Human Adipocytes

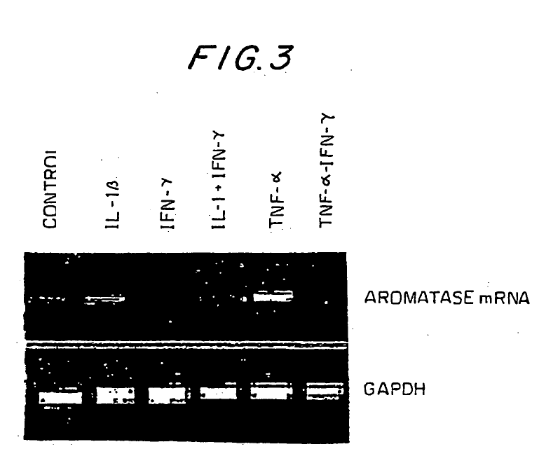

Adipocyte cultures were treated with IL-1-β, IFN-γ, TNF-α, and combinations of IL-1-β, and IFN-γ and TNF-α and IFN-γ. Subsequently, total RNA from cultured adipocytes was isolated using conventional methods. The reverse transcriptase polymerase chain reaction (RT-PCR) was performed using aromatase specific primers. The amplified aromatase cDNA fragments produced by RT-PCR were separated on an agarose gel. As shown in the FIG. 3, IL-1 and TNF alone induce transcription of aromatase mRNA compared to untreated control. However, the administration of IFN-γ inhibits the transcription of aromatase mRNA when given together IL-1 or TNF. The lower panel shows transcription of a constitutively expressed household gene, glcero-aldehyde phosphosphate dehydrogenase (GAPDH). Control lane shows the base level of aromatase mRNA expression in adipocytes.

### EXAMPLE 4

### IFN-γ Inhibited the Induction of Estradiol Production in Human Adipocytes by a Mixture of 6 Cytokines

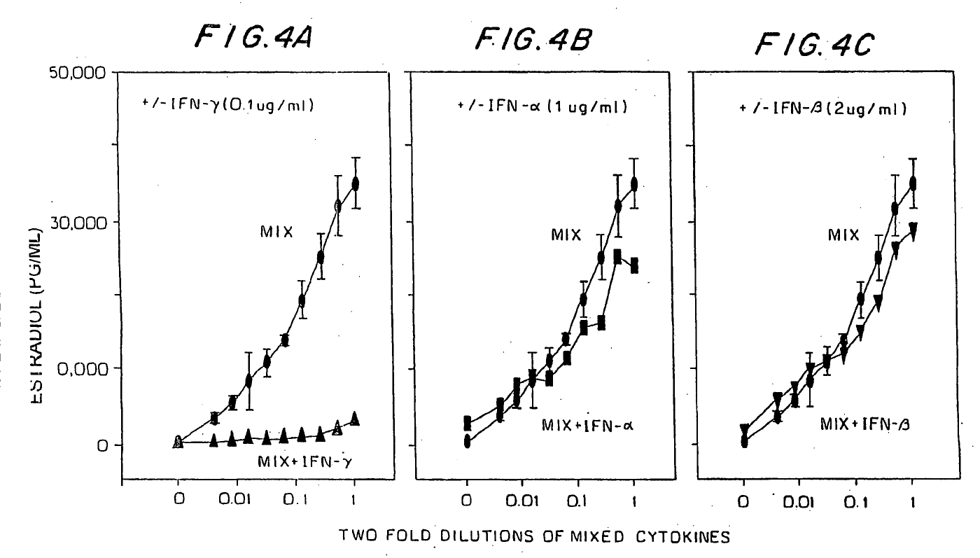

Human adipocytes (100,000 cells/well) were cultured for 72 hr in the presence or absence of different doses of the mix Cytokines (IL-1b, TNF-α, IL-6, IL-8, IL-11 and oncostatin M, 0.01 ug/ml/each—dilution as indicated) with and without IFN-γ (0.1 ug/ml), IFN-1a (1 ug/ml) or IFN-b (2 ug/ml). IFN-α was purchased from PBL while IFN-b was obtained from Serono. The amount of estradiol release in the conditioned media was determined by radioimmunoassay as described in Example 1. Each value represents the mean±S.E.M. of triplicate wells. Similar results were obtained in 3 additional experiments. The results show, as illustrated in FIG. 4, IFN-γ, but not IFN-α or IFN-β, inhibits adipocyte estradiol production.

### EXAMPLE 5

### IFN-γ Inhibited the Induction of Estradiol Production in Human Adipocytes by a Mixture of 15 Cytokines (IL-1a, IL-1b, TNF-α, TNF-β, IL-6, IL-8, IL-11, GH, IGF-1, LIF, SCF, FAS, TRAIL, OX40 L and Oncostatin M, 0.01 ug/ml/each)

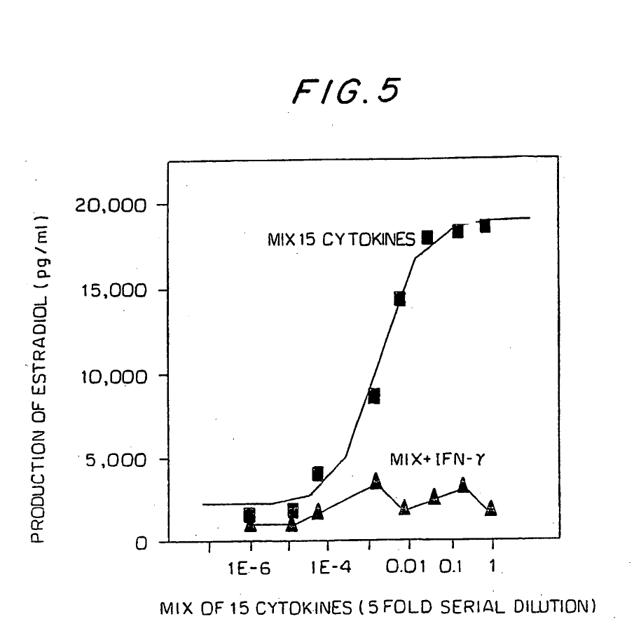

Human adipocytes (100,000 cells/well) obtained from Zen-Bio,Inc were cultured for 48 hr in the presence or absence of different doses of the cytokine mix (dilution as indicated) with and without IFN-γ (0.1 ug/ml). The amount of estradiol release in the conditioned media was determined by radioimmunoassay as described in Example 1. Each value represents the mean±S.E.M. of triplicate wells. Similar results were obtained in 3 additional experiments. As illustrated in FIG. 5, IFN-γ inhibited adipocyte estradiol production stimulated by the cytokine mix. These results indicate that the inhibitory effect of IFN-γ is not due to blocking the binding of the specific cytokines to their respective receptors.

### EXAMPLE 6

### IFN-γ Inhibited LPS Induction of Estradiol Production in Human Adipocytes

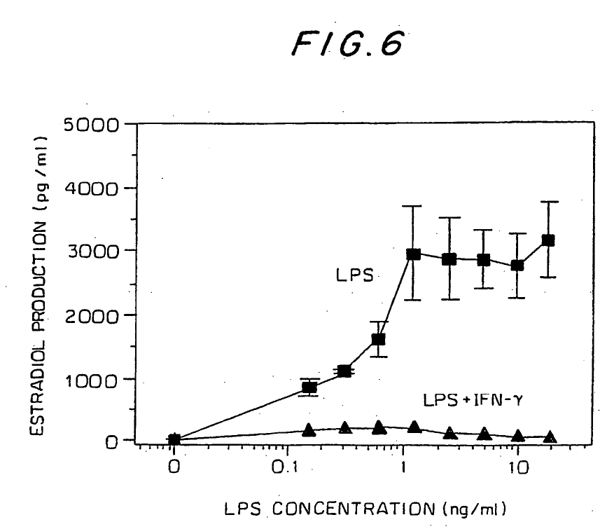

Human adipocytes (100,000 cells/well) obtained from Zen-Bio, Inc were cultured for 72 hr in the presence or absence of different doses of LPS (as indicated) with and without IFN-γ (0.1 ug/ml, LPS were purchased from Sigma). The amount of estradiol release in the conditioned media was determined by radioimmunoassay as described in Example 1. Each value represents the mean±S.E.M. of triplicate wells. Similar results were reproducible in 4 additional experiments using cells derived from separate patients. The results show, as illustrated in FIG. 6, that IFN-γ inhibits LPS stimulated estradiol production from adipocytes.

### EXAMPLE 7

### IFN-γ Inhibited Induction of Estradiol Production in Human Adipocytes by a Mix of PMA (5 ng/ml), LPS (10 ug/ml) and PGE2 (10 ug/ml)

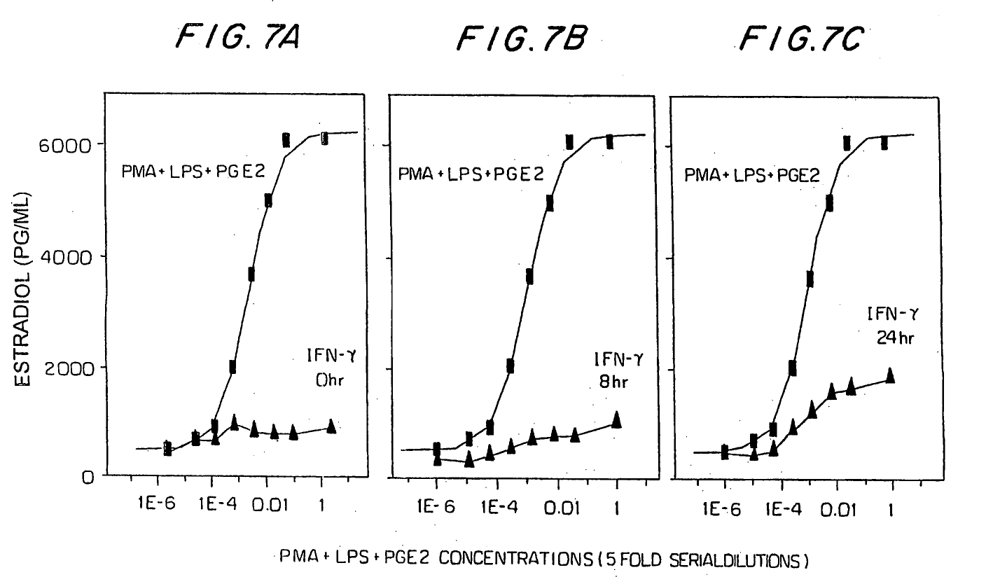

Human adipocytes (100,000 cells/well) obtained from Zen-Bio, Inc were cultured for 48 hr in the presence or absence of different doses of the mix LPS, PMA and PGE2 (dilution as indicated) with and without IFN-γ (0.1 ug/ml). IFN-γ was added at the same as the mix, 8 hr or 24 hr after the mix of the LPS, PMA and PGE2 which were purchased from Sigma. The amount of estradiol release in the conditioned media was determined by radioimmunoassay as described in Example 1. Each value represents the mean±S.E.M. of triplicate wells. The results show, as illustrated in FIG. 7, IFN-γ inhibits PMA, LPS and PGE2 stimulated estradiol production from adipocytes for zero to 24 hours.

### EXAMPLE 8

### IFN-γ Inhibited TRAIL, OX40L, IL-1b , IL-6, IL-11 and LIF Induction of Aromatase mRNA in Human Adipocytes

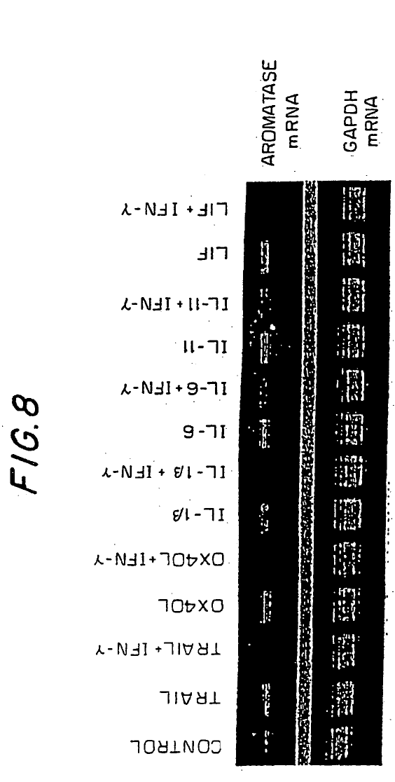

Human adipocytes (from one flask with 100% confluence) obtained from Zen-Bio,Inc were cultured for 72 hr in the presence or absence of 0.1 ug/ml of different Cytokines as indicated with and without IFN-γ (0.1 ug/ml, all Cytokines were purchased from R&D except FAS ligand, TRAIL and OX40 Ligands were obtained from Alexis). Total RNA was extracted for RT-PCR (30 cycles with 50 ng RNA/each sample) using human aromatase and GAPDH specific oligos (20 mers). The results show, as illustrated in FIG. 8, IFN-γ inhibits induced expression of aromatase mRNA by TRAIL, OX40, IL-1β, IL-6, IL-11 and LIF.

### EXAMPLE 9

### Cytokines (IL-6, IL-8, IL-11, OSM, GH and IGF-1) Stimulate the Production of Estradiol in Human Preadipocytes

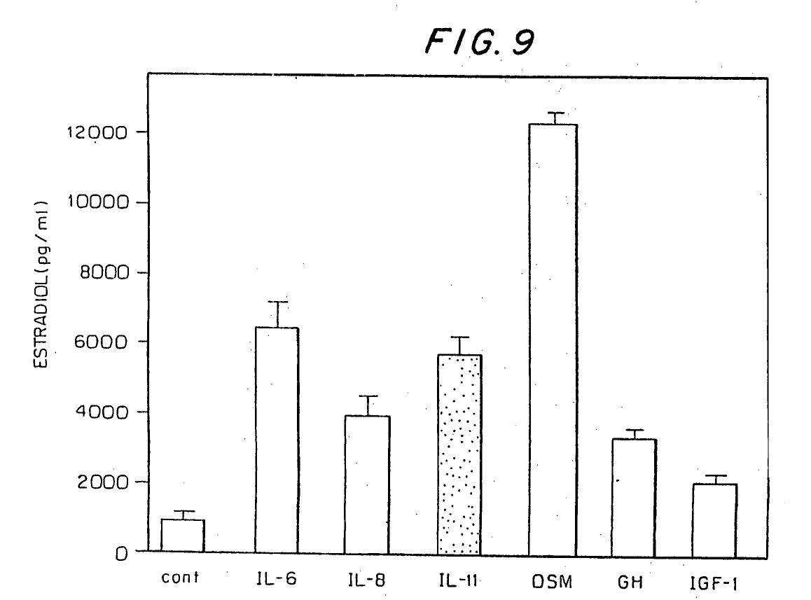

Human adipocytes (100,000 cells/well) were cultured for 72 hr in the presence of different Cytokines (IL-6 (0.5 ug/ml), IL-8 (0.5 ug/ml), IL-11 (0.5 ug/ml), oncostatin M (0.5 ug/ml), GH (5 ug/ml) and IGF-1 (5 ug/ml). The amount of estradiol release in the conditioned media was determined by radioimmunoassay as described in Example 1. Each value represents the mean±S.E.M. of triplicate wells. The results show, as illustrated in FIG. 9, that cytokines, IL-6, IL-8, IL-11, OSM, GH and IGF-1, each stimulate estradiol production in human preadipocytes.

### EXAMPLE 10

### Monoclonal Antibodies to Oncostatin M Inhibit the Induction of Estradiol Production in Human Adipocytes by Oncostatin M

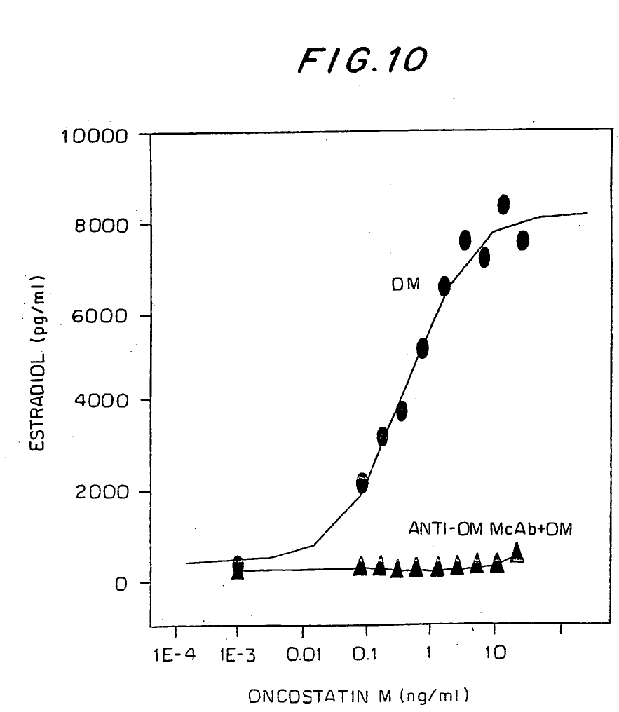

Human adipocytes (100,000 cells/well) were cultured for 72 hr in the presence Oncostatin M (concentation as indicated) with and without monoclonal antibodies to Oncostatin M (200 ug/ml). The amount of estradiol release in the conditioned media was determined by radioimmunoassay as described in Example 1. Each value represents the mean±S.E.M. of triplicate wells. The results show, as illustrated in FIG. 10, monoclonal antibodies to Oncostatin M inhibit adipocyte estradiol production stimulated by Oncostatin M.

### EXAMPLE 11

### Monoclonal Antibodies to IL-11 Inhibit the Induction of Estradiol Production in Human Adipocytes by IL-11

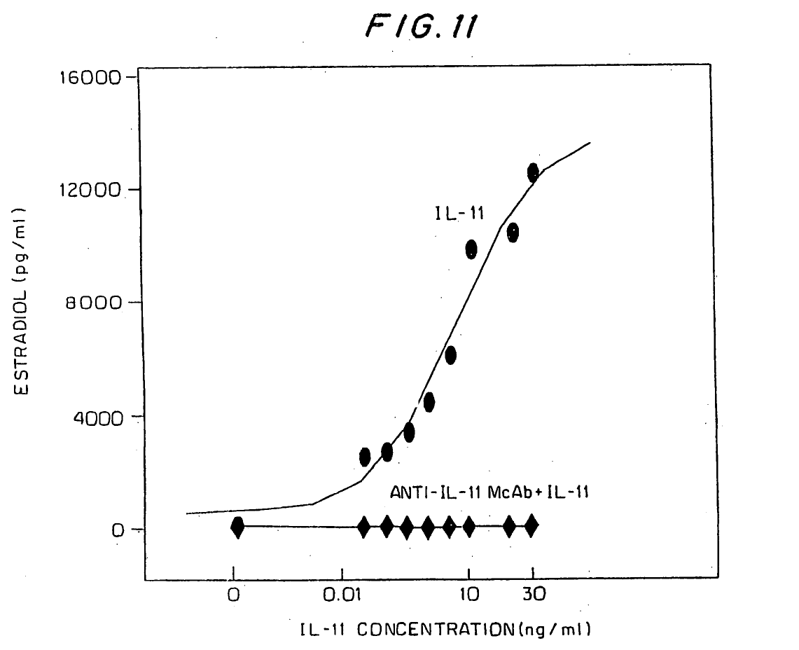

Human adipocytes (100,000 cells/well) were cultured for 72 hr in the presence IL-11 (concentration as indicated) with and without monoclonal antibodies to IL-11 (200 ug/ml). The amount of estradiol release in the conditioned media was determined by radioimmunoassay as described in Example 1. Each value represents the mean±S.E.M. of triplicate wells. The results show, as illustrated in FIG. 11, monoclonal antibodies to IL-11 inhibit adipocyte estradiol production stimulated by IL-11.

### EXAMPLE 12

### Interferon Alpha/Beta Receptor (IFNAR) Inhibits the IFN-β Induction of Estradiol Production in Human Adipocytes

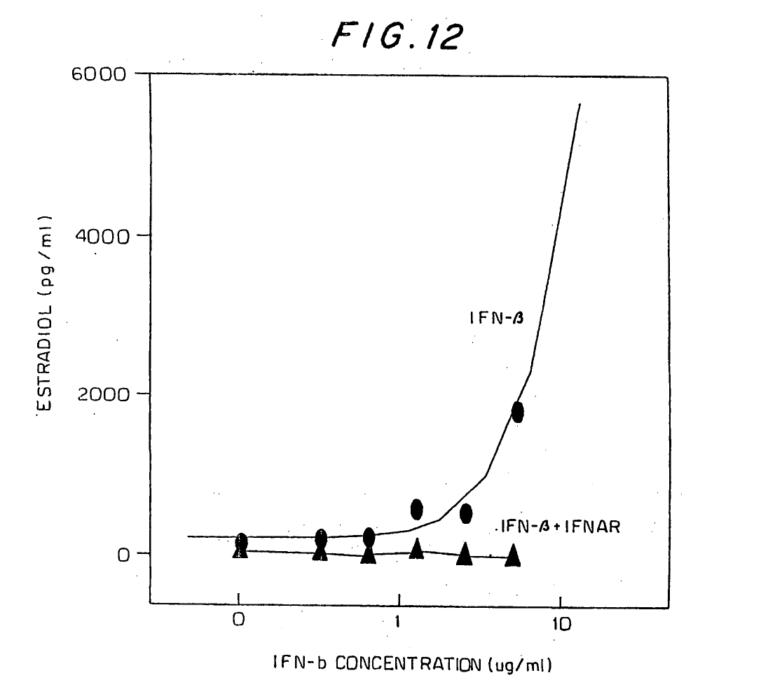

Human adipocytes (100,000 cells/well) were cultured for 72 hr in the presence IFN-β (concentrations as indicated) with and without IFNAR (4 ug/ml). The amount of estradiol release in the conditioned media was determined by radioimmunoassay as described in Example 1. Each value represents the mean±S.E.M. of triplicate wells. The results show, as illustrated in FIG. 12, IFNAR inhibits adipocyte estradiol production stimulated by IFN-β.

### EXAMPLE 13

### IL-1 Receptor Antagonist (IL-1RA) Inhibits the IL-1α and IL-1β Induction of Estradiol Production in Human Adipocytes

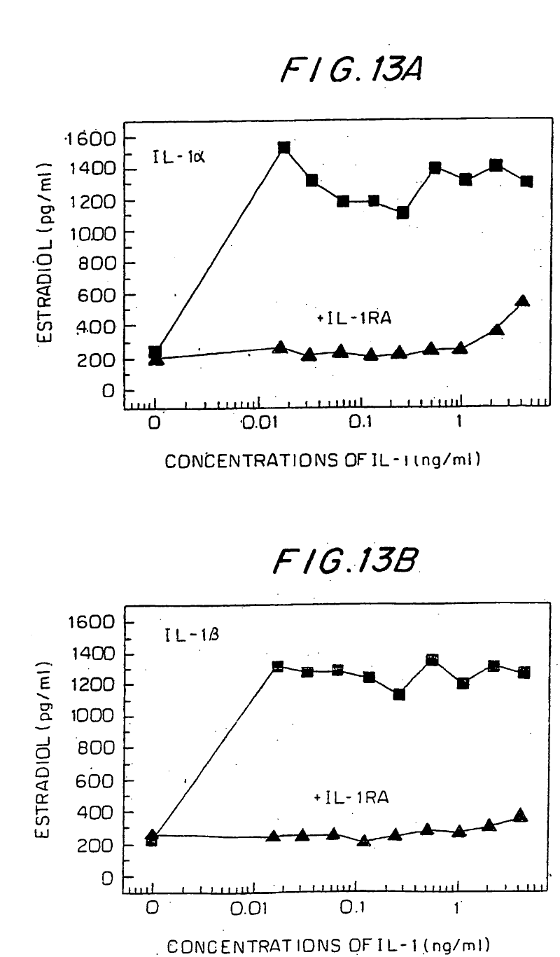

Human adipocytes (100,000 cells/well) were cultured for 72 hr in the presence IL-1α and IL-1β (concentrations as indicated) with and without IL-1RA (4 ug/ml). The amount of estradiol release in the conditioned media was determined by radioimmunoassay as described in Example 1. Each value represents the mean±S.E.M. of triplicate wells. The results show, as illustrated in FIG. 13, IL-1RA inhibits adipocyte estradiol production stimulated by IL-1α and IL-1β.

The preceding examples are to be evaluated as illustrative and are not intended to limit the scope of the invention.

All publications and patent applications cited in this specification are herein incorporated by reference as if each individual publication or patent application were specifically and individually indicated to be incorporated by reference. Although the foregoing invention has been described in some detail by way of illustration and an example for purposes of clarity of understanding, it will be readily apparent to those of ordinary skill in the art in light of the teachings of this invention that certain changes and modifications may be made thereto without departing from the spirit or scope of the appended claims.

1. A method of treating a woman suffering from or susceptible to endometriosis, comprising administering to the woman an effective amount of interferon-gamma (IFN-γ).

2. A method of treating a woman suffering from or susceptible to endometriosis, comprising administering to the woman an effective amount of a cytokine antagonist.

3. A method of treating a woman suffering from or susceptible to endometriosis comprising administering a therapeutically effective amount of a combination of IFN-γ and a cytokine antagonist.

4. A method of treating a woman suffering from or susceptible to endometriosis comprising administering a therapeutically effective amount of IFN-γ in combination with an anti-estrogenic agent.

5. A method of treating a woman suffering from or susceptible to endometriosis comprising administering a therapeutically effective amount of a cytokine antagonist in combination with an anti-estrogenic agent.

6. A method of treating a woman suffering from or susceptible to endometriosis comprising administering a therapeutically effective amount of IFN-γ and a cytokine antagonist in combination with an anti-estrogenic agent.

7. The method of claim 4, wherein the anti-estrogenic agent is an aromatase inhibitor.

8-12. (canceled)

13. An article of manufacture comprising:
i) a packaging material comprising a label which indicates said pharmaceutical may be administered, for a sufficient term at an effective dose, for treating or preventing endometriosis in a woman; and
ii) a pharmaceutical agent contained within said packaging material, wherein said pharmaceutical agent comprises IFN-γ together with a pharmaceutical acceptable carrier

14. An article of manufacture comprising:
i) a packaging material comprising a label which indicates said pharmaceutical may be administered, for a sufficient term at an effective dose, for treating or preventing endometriosis in a woman; and
ii) a pharmaceutical agent contained within said packaging material, wherein said pharmaceutical agent comprises a cytokine antagonist together with a pharmaceutical acceptable carrier

15. An article of manufacture comprising:
i) a packaging material comprising a label which indicates said pharmaceutical may be administered, for a sufficient term at an effective dose, for treating or preventing endometriosis in a woman; and
ii) a pharmaceutical agent contained within said packaging material, wherein said pharmaceutical agent comprises a combination of IFN-γ and a cytokine antagonist together with a pharmaceutical acceptable carrier

16. The method of claim 2, wherein the cytokine antagonist is selected from the group consisting of antagonists to tumor necrosis factor-related apoptosis-inducing ligand, OX40 Ligand, lymphotoxin alpha, interleukin 1 alpha, interleukin-1 beta, interleukin-6, interleukin-8, interleukin-11, leukemia inhibitory factor, Fas Ligand, oncostatin M, interferon alpha, interferon beta, insulin growth factor-1, stem cell factor and human growth hormone.

17. The method of claims 3, wherein the cytokine antagonist is an antagonist to tumor necrosis factor.

18. The method of claim 5, wherein the anti-estrogenic agent is an aromatase inhibitor.

19. The method of claim 6, wherein the anti-estrogenic agent is an aromatase inhibitor.

20. The method of claims 5, wherein the cytokine antagonist is an antagonist to tumor necrosis factor.

21. The method of claims 6, wherein the cytokine antagonist is an antagonist to tumor necrosis factor.

22. The method of claim 3, wherein the cytokine antagonist is selected from the group consisting of antagonist to tumor necrosis factor-related apoptosis-inducing ligand, OX40 Ligand, lymphotoxin alpha, interleukin 1 alpha, interleukin-1 beta, interleukin-6, interleukin-8, interleukin-11, leukemia inhibitory factor, Fas Ligand, oncostatin M, interferon alpha, interferon beta, insulin growth factor-1, stem cell factor and human growth hormone.

23. The method of claim 5, wherein the cytokine antagonist is selected from the group consisting of antagonists to tumor necrosis factor-related apoptosis-inducing ligand, OX40 Ligand, lymphotoxin alpha, interleukin 1 alpha, interleukin-1 beta, interleukin-6, interleukin-8, interleukin-11, leukemia inhibitory factor, Fas Ligand, oncostatin M, interferon alpha, interferon beta, insulin growth factor-1, stem cell factor and human growth hormone.

24. The method of claim 6, wherein the cytokine antagonist is selected from the group consisting of antagonists to tumor necrosis factor-related apoptosis-inducing ligand, OX40 Ligand, lymphotoxin alpha, interleukin 1 alpha, interleukin-1 beta, interleukin-6, interleukin-8, interleukin-11, leukemia inhibitory factor, Fas Ligand, oncostatin M, interferon alpha, interferon beta, insulin growth factor-1, stem cell factor and human growth hormone.

25. The method of claim 14, wherein the cytokine antagonist is selected from the group consisting of antagonists to tumor necrosis factor-related apoptosis-inducing ligand, OX40 Ligand, lymphotoxin alpha, interleukin 1 alpha, interleukin-1 beta, interleukin-6, interleukin-8, interleukin-11, leukemia inhibitory factor, Fas Ligand, oncostatin M, interferon alpha, interferon beta, insulin growth factor-1, stem cell factor and human growth hormone.

26. The method of claim 15, wherein the cytokine antagonist is selected from the group consisting of antagonists to tumor necrosis factor-related apoptosis-inducing ligand, OX40 Ligand, lymphotoxin alpha, interleukin 1 alpha, interleukin-1 beta, interleukin-6, interleukin-8, interleukin-11, leukemia inhibitory factor, Fas Ligand, oncostatin M, interferon alpha, interferon beta, insulin growth factor-1, stem cell factor and human growth hormone.
